(19) United States
(12) Patent Application Publication
(10) Pub. No.: US 2017/0136204 A1
(43) Pub. Date: May 18, 2017

(54) CONSUMER MIGRAINE RELIEF DEVICE FOR USE WITH BEVERAGE CONTAINERS

(71) Applicant: Dak Brandon Steiert, Edwards, CO (US)

(72) Inventor: Dak Brandon Steiert, Edwards, CO (US)

(21) Appl. No.: 14/941,567

(22) Filed: Nov. 14, 2015

(51) Int. Cl. A61M 16/12; A61M 15/08; A61M 16/06; A61M 16/20

(52) U.S. Cl. CPC A61M 16/12; A61M 16/20; A61M 15/08; A61M 16/0666; A61M 2202/0225; A61M 2202/0275; A61M 2202/0283

(57) ABSTRACT

The migraine treatment device of the preferred embodiments includes a threaded fitting designed to thread onto a beverage container; a valve coupled to the threaded fitting; a nozzle coupled to the valve, where the nozzle opening is smaller than the size of a human nostril; where at least some of the gas in the beverage container is one or more of: a) carbon dioxide, b) nitric oxide, c) nitrous oxide, d) an acidic gas, e) a gas that reacts with moisture to create acid, and f) an acidic vapor. The migraine treatment device is preferably designed to deliver gas into the nasal passageway of a user, where the gas is capable of reacting with moisture to create an acid so that the acid activates a sensation in the nerves of the nasal passageway, causing a reduction in a user's discomfort due to at least one of headaches and migraine headaches.

### BRIEF DESCRIPTION OF THE FIGURES

FIG. 1 is a schematic representation of the system of the preferred embodiments, showing the migraine treatment device relative to a user's nostril.

FIG. 2 is a schematic representation of the system of the preferred embodiments, where the migraine treatment device is threaded onto a consumer beverage container.

FIG. 3 is a schematic representation of the system of the preferred embodiments, where the migraine treatment device is threaded onto a plastic soda bottle.

FIG. 4 is a schematic representation of the system of the preferred embodiments, where the migraine treatment device is threaded onto a plastic soda bottle containing a carbonated liquid, where the nozzle is held against a user's nostril, where the device delivers gas into the user's nasal passageway.

FIG. 5 is a schematic representation of the system of the preferred embodiments, where a solid and liquid react to release carbon dioxide, where the solid may be at least one of an effervescent tablet and any other suitable liquid.

FIG. 6 is a schematic representation of the system of the preferred embodiments, where a nasal mask is attached to the valve, shown with the nasal mask placed over a user's nose and at least partially sealing against the user's facial anatomy

FIG. 7 is a schematic representation of the system of the preferred embodiments, where a nasal mask is attached to the valve, where the migraine treatment device is threaded onto a plastic soda bottle containing a carbonated beverage.

FIG. 8 is a schematic representation of the system of the preferred embodiments, where the valve is a spring loaded, normally closed, push button valve, and where the migraine treatment device is threaded onto a plastic soda bottle.

FIG. 9 is a schematic representation of the system of the preferred embodiments, where the valve is a spring loaded, normally closed, push button valve, and where the migraine treatment device is threaded onto a plastic soda bottle, where a section of flexible tubing is attached to the valve and attached at the distal end of the tube to the nozzle assembly.

FIG. 10 is a schematic representation of the system of the preferred embodiments, where the valve is a spring loaded, normally closed, push button valve, and where the migraine treatment device is threaded onto a plastic soda bottle, where a section of flexible tubing is attached to the valve and attached at the distal end of the tube to a nasal mask assembly.

### DESCRIPTION OF THE PREFERRED EMBODIMENTS

The following description of the preferred embodiments of the invention is intended to enable someone skilled in the prior art to make and use this invention, but is not intended to limit the invention to these preferred embodiments.

### 1. First Preferred Embodiment

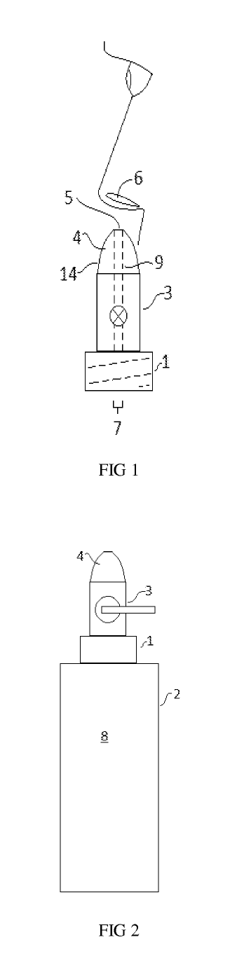

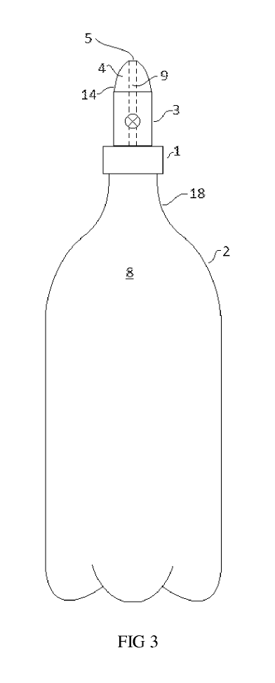

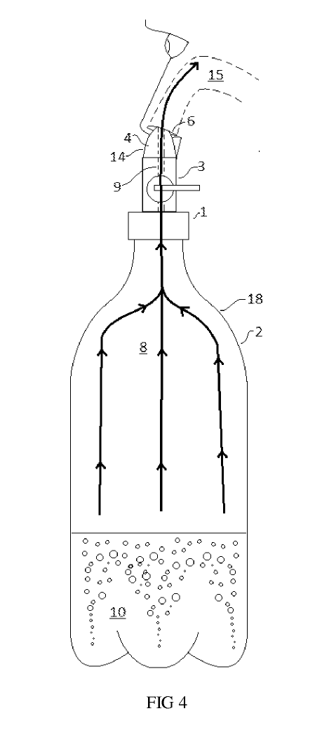

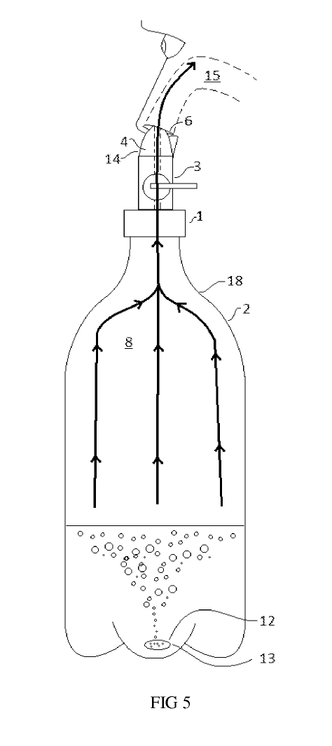

As shown in FIG. 1, the migraine treatment device of the preferred embodiments includes a threaded fitting 1 designed to thread onto a beverage container 2; a valve 3 coupled to the threaded fitting 1; a nozzle 4 coupled to the valve 3, where the nozzle 4 opening 5 is smaller 7 than the size of a human nostril 6; where at least some of the gas 8 in the beverage container 2 is one or more of: a) carbon dioxide, b) nitric oxide, c) nitrous oxide, d) an acidic gas, e) a gas that reacts with moisture to create acid, and f) an acidic vapor. The migraine treatment device is preferably designed to deliver gas 8 into the nasal passageway 15 of a user, where the gas 8 is capable of reacting with moisture to create an acid so that the acid activates a sensation in the nerves of the nasal passageway 15, causing a reduction in a user's discomfort due to at least one of headaches and migraine headaches.

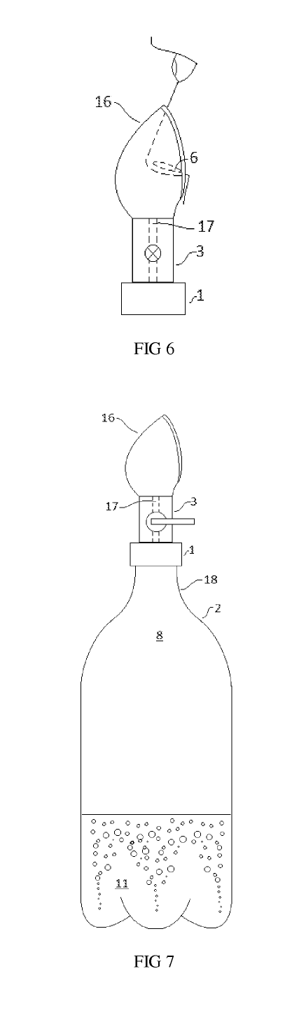

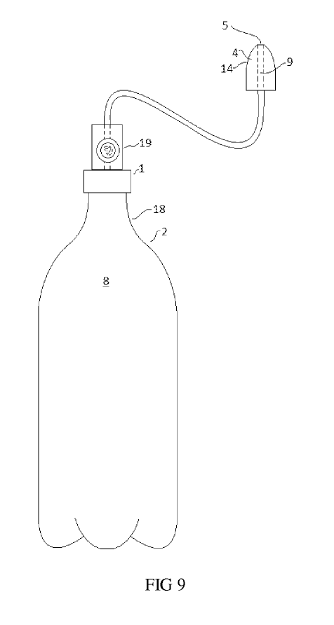

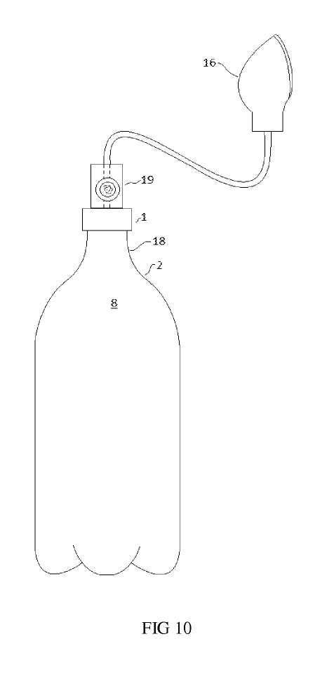

As shown in FIG. 1, the nozzle 4 has an opening 5 that is smaller 7 than the size of a human nostril 6. The nozzle 4 is designed to be held at least one of near and against the user's nostril 6 so that the migraine treatment device can deliver gas 8 into the user's nasal passageway 15. The exterior surface 14 of the nozzle 4 is preferably designed to at least partially seal against a user's nostril 6 when held against a user's nostril 6. The exterior surface 14 of the nozzle 4 is preferably at least one of) a conical shape, b) a frusto-conical shape, c) a parabolic shape, and d) a hemispherical shape. The nozzle 4 may, however, have any suitable shape allowing gas 8 to be delivered into a user's nasal passageway 15. As shown in FIG. 9, in another preferred variation a flexible tube may be coupled to the valve 3, and the nozzle 4 assembly may be coupled to the distal end of the flexible tube. This may be convenient for a user, and may allow a user to leave the beverage container 2 on a support surface while delivering gas 8 into their nasal passageway 15. In another preferred variation, a fitting that can fit onto the opening of a consumer beverage container and seal and attach to the opening without threads by any suitable means, such as a clamp and seal, may be used instead of a threaded fitting. As shown in FIGS. 6 and 7, in an alternative preferred variation, a nasal mask 16 is coupled to the valve 3, where the nasal mask 16 is preferably designed to go over at least part of a user's nose. In this preferred variation, the nasal mask 16 is preferably designed to at least partially seal against a user's facial anatomy. A user's facial anatomy preferably includes one or more of a user's nose, a user's cheeks, a user's lips, a user's nostrils, and any other suitable portion of a user's face. As shown in FIG. 10, in another preferred variation a flexible tube may be connected to the valve 3, and the nasal mask 16 assembly may be connected to the distal end of the flexible tube. The nasal mask 16 may, however, have any suitable design for delivering gas 8 into a user's nasal passageway 15. The migraine treatment device may, however, have any suitable interface for delivering gas 8 into a user's nasal passageway 15.

The gas 8 in the beverage container 2 is preferably at least in part one of: a) carbon dioxide, b) nitric oxide, c) nitrous oxide, d) an acidic gas, e) a gas that reacts with moisture to create acid, and f) an acidic vapor. As described in US Pat. No. 8,096,968, delivering at least one of these gases to a user's nasal passageway 15 may be beneficial in treating at least one of 1) headaches and 2) migraine headaches. In a preferred variation, the gas 8 is carbon dioxide. In a preferred variation, the carbon dioxide gas 8 is released from a carbonated liquid 10. In a preferred variation, the carbonated liquid 10 is a carbonated beverage 11. In a preferred variation, a user can take a carbonated beverage 11, ensure that some of the beverage container 2 is empty to provide a volume to act as a reservoir for gas 8, preferably ensuring that between 30% and 80% of the beverage container is free of liquid and the gas may occupy the 30% to 80% of the beverage container volume, then the user may thread the migraine treatment device onto the opening of the consumer beverage container 2 and ensure that it seals onto the consumer beverage container 2, then the user may shake the bottle so that the empty space fills with carbon dioxide gas 8 released from the carbonated beverage 11 to a pressure of greater than 7 psi to 10 psi above atmospheric pressure, then the user may hold at least one of the nozzle 4 and the nasal mask 16 so that the gas 8 can be delivered into at least one of the user's nostrils, and then the user may open the valve 3 and inhale the carbon dioxide into the user's nasal passageway 15. The migraine treatment device may, however, be used in any suitable manner. In a preferred variation, the consumer beverage container 2 is a plastic soda bottle 18. Preferably the consumer beverage container 2 is a two liter bottle or smaller. In a preferred variation, the consumer beverage container 2 is a soda bottle 18 with at least a 750 milliliter volume. In another preferred variation, the consumer beverage container 2 is a soda bottle 18 with at least a 1 liter volume. In the preferred variation where the consumer beverage container 2 is a soda bottle 18, the threaded fitting 1 has threads designed to thread onto a plastic soda bottle 18. In this preferred variation, the threaded fitting 1 preferably further includes a compliant polymer seal to seal against the threaded opening of the consumer beverage container 2. If the soda bottle 18 is too small, the volume in the bottle does not provide a large enough gas 8 reservoir to deliver enough gas 8 to effectively treat at least one of headaches and migraine headaches, and the volume of carbonated beverage 11 is not high enough to provide enough carbon dioxide to create effective treatment. In another variation, a smaller consumer beverage container 2 may be used if the user follows an adjusted method of use. The consumer beverage container 2 may, however, be any suitable type of container. The consumer beverage container 2 may, however, be any suitable size. As shown in FIG. 5, in another preferred variation carbon dioxide is created in the consumer beverage container 2 by the reaction of a solid 12 with a liquid. In one preferred variation, carbon dioxide is created in the consumer beverage container 2 by the reaction of an effervescent tablet 13 with water. In another preferred variation, carbon dioxide is created by the reaction of at least one of sodium bicarbonate and sodium carbonate and at least one of an acid and water. The source of carbon dioxide may, however, be any suitable source. The gas 8 may, however, be any gas suitable for interacting with the sinus nerves and having an effect on the capillary system in the user's nasal passageway 15.

As shown in FIG. 1, in a preferred variation at least one of a) the valve 3, b) the threaded fitting 1, and c) the nozzle 4 has an opening sized to constrict the flow of gas 8 to a rate causing the release of the gas 8 in a consumer beverage container 2 to last longer than 0.1 seconds (one tenth of a second), wherein the consumer beverage container 2 has a volume between 750 milliliters and two liters, wherein the source of the gas 8 is release from a consumer beverage, wherein the gas pressure at the beginning of the release is at least 10 pounds per square inch above atmospheric pressure, and where at least 60% of the volume of the beverage container is free from liquids and may be occupied by the gas 8. In this preferred variation, the release of the gas 8 is measured until the gas 8 has been released from the consumer beverage container 2 to the point that the pressure has equalized with atmospheric pressure, where the pressure at the beginning of the gas 8 release was no more at least 10 psi above atmospheric pressure (ten psig). The gas release times will be shorter if any of the following conditions exist at the time of release: the beverage container is smaller, the starting pressure is lower, or the beverage container is filled with more than 60% liquid or other obstructions, preventing the gas from occupying at least 60% of the volume of the container at the beginning of the release, and the temperature is lower than 72 degrees Fahrenheit, so the invention is preferably made to create the preferred release times or longer under the stated conditions, rather than under conditions which might otherwise reduce release times. These stated conditions roughly correspond to one preferred variation of the practical use of the system of the preferred embodiments. In a preferred variation the gas 8 is delivered too quickly to the user's nasal passageway 15, the therapeutic effect is greatly diminished to the point that the migraine treatment device will not be significantly effective in treating at least one of headaches and migraine headaches, so in this preferred variation, slowing the release of the gas is necessary to the functioning of the migraine treatment device. In another preferred variation, the gas 8 is constricted by at least one of a) the valve, b) the threaded fitting, and c) the nozzle to ensure that the release of the gas 8 lasts at least 0.15 seconds (fifteen hundredths of a second). In another preferred variation, the gas 8 is constricted by at least one of a) the valve, b) the threaded fitting, and c) the nozzle to ensure that the release of the gas 8 lasts at least 0.25 seconds (twenty-five hundredths of a second). The migraine treatment device must appropriately balance sufficient delivery of gas 8 with sufficient length of time of gas 8 delivery. In a preferred variation, the gas 8 release is constricted by including at least one constriction opening into at least one of a) the valve, b) the threaded fitting, the c) the nozzle, and d) the gas passage 17 delivering gas 8 into the nasal mask 16, this constriction opening can be at any point in the gas passageway 9 leading from the beverage container interior through at least one of the nozzle and the gas passage 17 delivering gas 8 into the nasal mask 16. In this preferred variation, the at least one constriction opening has an area of less than 0.0125 square inches. In another preferred variation, the constriction opening has an area of less than 0.004 square inches. In another preferred variation, the constriction opening has an area of less than 0.001 square inches. The constriction opening may, however, have any suitable opening area to slow the rate of release of the gas to achieve a suitable release time. The valve, threaded fitting, and nozzle may, however, have any suitable design to provide suitable delivery of gas. The gas release time may, however, be any suitable length. In another preferred variation, however, the gas release time may be a less important factor in determining the therapeutic effects of the migraine treatment device due to factors including the total quantity of gas released, the exact nature of delivery into the nasal passageway, the temperature of the gas released, the chemical composition of the gas released, and any other suitable factors altering the therapeutic effect of the gas delivery.

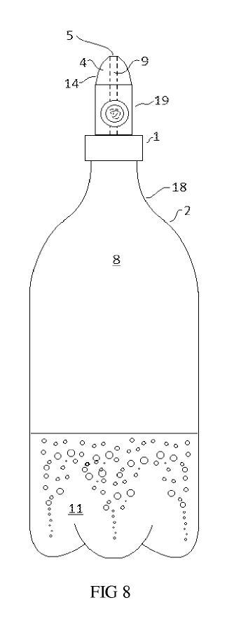

As shown in FIG. 8, in a preferred variation the valve 3 may be a spring biased, normally closed, push button valve 19. In this preferred variation, the valve 3 preferably allows gas flow when pushed and closes when released. This valve 3 is preferably adapted to allow a user to easily operate the valve 3 with one hand. As shown in FIGS. 9 and 10, in a preferred variation, the spring biased, normally closed, push button valve 19 may be attached to the flexible tube in order to provide a system that is convenient and comfortable to use. In another preferred variation, at least one of a ball valve, a needle valve, and any other suitable valve may be used. The valve 3 may, however, be any suitable gas flow control device. There may, however, be no flexible tube used in the device.

As a person skilled in the art will recognize from the previous detailed description and from the figures and claims, modifications and changes can be made to the preferred embodiments of the invention without departing from the scope of this invention defined in the following claims.

I claim:

1) A migraine treatment device comprising a threaded fitting adapted to thread onto a beverage container; a valve coupled to the threaded fitting; a nozzle coupled to the valve, wherein the nozzle opening is smaller than the size of a human nostril; wherein at least some of the gas in the beverage container is at least one of: a) carbon dioxide, b) nitric oxide, c) nitrous oxide, d) an acidic gas, e) a gas that reacts with moisture to create acid, and f) an acidic vapor.

2) The migraine treatment device of claim 1 wherein the gas comprises carbon dioxide.

3) The migraine treatment device of claim 2 wherein the carbon dioxide is released from a carbonated liquid.

4) The migraine treatment device of claim 2 wherein the carbon dioxide is released by a chemical reaction between a liquid and a solid, wherein the chemical reaction is carried out inside the beverage container.

5) The migraine treatment device of claim 4 wherein the chemical reaction is between an effervescent tablet and water.

6) The migraine treatment device of claim 3 wherein the carbonated liquid is a carbonated beverage.

7) The migraine treatment device of claim 3 wherein the nozzle is shaped the interface with a human nostril.

8) The migraine treatment device of claim 7 wherein the nozzle has an exterior surface with at least one of a) a conical shape, b) a frusto-conical shape, c) a parabolic shape, and d) a hemispherical shape.

9) The migraine treatment device of claim 8 wherein the exterior of the nozzle is sized to at least partially seal off a human nostril when held against a human nostril.

10) The migraine treatment device of claim 3 wherein the nozzle is adapted to deliver gas into the nasal passageway of a user.

11) The migraine treatment device of claim 9 wherein the carbon dioxide is released by a chemical reaction between a liquid and a solid, wherein the chemical reaction is carried out inside the beverage container.

12) The migraine treatment device of claim 11 wherein the chemical reaction is carried out by an effervescent tablet and water.

13) The migraine treatment device of claim 9 wherein the at least one of a) the valve, b) the threaded fitting, and c) the nozzle has an opening sized to constrict the flow of gas to a rate causing the release of the gas in a beverage container to last longer than 0.1 seconds, wherein the beverage container has a volume between 750 milliliters and two liters, wherein at least 60% of the volume of the beverage container is volume is free of liquid and occupied by the gas, wherein the gas pressure is at least 10 pounds per square inch above atmospheric pressure, and the temperature is at least 72 degrees Fahrenheit.

14) The migraine treatment device of claim 10 wherein the at least one of a) the valve, b) the threaded fitting, and c) the nozzle has an opening sized to constrict the flow of gas to a rate causing the release of the gas in the beverage container to last longer than 0.15 seconds, wherein the beverage container has a volume between 750 milliliters and two liters, wherein at least 60% of the volume of the beverage container is volume is free of liquid and occupied by the gas, wherein the gas pressure is at least 10 pounds per square inch above atmospheric pressure, and the temperature is at least 72 degrees Fahrenheit.

15) A migraine treatment device comprising a threaded fitting adapted to thread onto a beverage container; a valve coupled to the threaded fitting; a nasal mask coupled to the valve wherein the nasal mask is designed to fit over at least part of a human nose and at least partially seal against the user's facial anatomy; wherein a gas passage delivers gas from the nozzle into the nasal mask internal cavity; wherein the gas is at least one of: a) carbon dioxide, b) nitric oxide, c) nitrous oxide, d) an acidic gas, e) a gas that reacts with moisture to create acid; wherein the beverage container is a plastic soda bottle, wherein the at least one of a) the valve, b) the threaded fitting, and c) the gas passage delivering gas into the nasal mask has an opening sized to constrict the flow of gas to a rate causing the release of the gas in the beverage container to last longer than 0.15 seconds, wherein the beverage container has a volume between 750 milliliters and two liters, wherein at least 60% of the volume of the beverage container is volume is free of liquid and occupied by the gas, wherein the gas pressure is at least 10 pounds per square inch above atmospheric pressure.

16) The migraine treatment device of claim 12 wherein the at least one of a) the valve, b) the threaded fitting, and c) the nozzle has an opening sized to constrict the flow of gas to a rate causing the release of the gas in the beverage container to last longer than 0.25 seconds.

17) The migraine treatment device of claim 2 wherein the beverage container is a plastic soda bottle.

18) The migraine treatment device of claim 3 wherein the beverage container is a plastic soda bottle.

19) The migraine treatment device of claim 5 wherein the beverage container is a plastic soda bottle, wherein the at least one of a) the valve, b) the threaded fitting, and c) the nozzle has an opening sized to constrict the flow of gas to a rate causing the release of the gas in the beverage container to last longer than 0.25 seconds.

20) The migraine treatment device of claim 10 wherein the beverage container is a plastic soda bottle.
